(19) United States
(12) Patent Application Publication
(10) Pub. No.: US 2008/0138419 A1
(43) Pub. Date: Jun. 12, 2008

(54) Method for preparing an orally administrable formulation for controlled release

(75) Inventors: Chao-Wei Liao, Chu Nan Town (TW); Peggy Lin, Chu Nan Town (TW); Chung-Nan Weng, Chu Nan Town (TW)

Correspondence Address: LADAS & PARRY LLP, 26 WEST 61ST STREET, NEW YORK, NY, 10023, US

(73) Assignee: ANIMAL TECHNOLOGY INSTITUTE TAIWAN

(21) Appl. No.: 11/708,397

(22) Filed: Feb. 20, 2007

Related U.S. Application Data
(63) Continuation-in-part of application No. 10/414,177, filed on Apr. 15, 2003, now abandoned.

(51) Int. Cl. A61K 9/10; A61K 47/38; A61K 47/30; A61K 35/66; A61K 38/02; A61K 38/43; A61K 39/00

(52) U.S. Cl. 424/489; 514/781; 514/772.3; 424/93.1; 514/2; 424/94.1; 424/184.1

(57) ABSTRACT

The present invention provides a method for preparing an orally administrable formulation comprising a biologically active ingredient for controlled release in a neutral or basic environment, which comprises the steps of: (a) dispersing powder ethylcellulose with an average diameter from about 0.1 μm to about 300 μm in an aqueous solution to provide an aqueous dispersion, wherein the aqueous dispersion is substantially free of detergent; (b) mixing the biologically active ingredient and the aqueous dispersion obtained in step (a) to provide a mixture; and (c) spray-drying the mixture obtained in step (b) for about 10 seconds to about 2 minutes in a drying chamber at a chamber temperature of about 45° C. to about 100° C. to obtain the orally administrable formulation. An orally administrative formulation prepared by the method of the invention is also provided.

### BACKGROUND OF THE INVENTION

1. Field of the Invention

The invention mainly relates to a method for preparing an orally administrable formulation for controlled release of a biologically active ingredient.

2. Description of the Related Art

Biologicals (biological composition) such as microorganisms, enzymes and proteins, are used as vaccines, and heat liable pharmaceutical compositions for humans and animals. Generally, the biologicals are usually delivered by injection to maintain a good biological activity. However, the cost of injection and the resistance of the subjects to be administrated limit the applications of the biologicals.

To overcome the limitations of injection administration, orally administrable formulations are developed. However, it is difficult and complex to prepare an effective formulation for an oral administration of the biologicals. For instance, the biologicals administrated orally are absorbed and targeted or reacted in the intestines. In other words, the biologicals should be encapsulated with an enteric material for delivering and controlling release in the intestines to avoid acid damage in the stomach. Therefore, the encapsulation process of the heat-liable biologicals is normally conducted by a coating process. The coating process, either with an organic solvent or an aqueous system, has been applied extensively in pharmaceutical systems. The impact of various external and internal forces against solvent systems, such as the explosive hazard and environmental pollution, has revitalized an interest in aqueous coating alternatives. However, using water as a solvent, more time or energy is required for evaporation. In a conventional preparation for a pre-oral enteric formulation containing the biologicals, the material suitable for an enteric coating is used, such as cellulose acetate phthalate (CAP), methyl methacrylate methacrylic acid copolymer, hydroxy propyl methyl cellulosephthalate (HPMCP), and polyvinyl acetate phthalate (PVAP). Such macromolecular materials are dissolved in a neutral or basic environment to release the biologically active ingredient encapsulated therein, but not in an acid environment. However, the encapsulation process must be conducted at a high temperature or in harsh organic solvent, under which the biological activity of the biologically active ingredient is decreased dramatically. Besides, the cost for the coating process is very high, especially for animal use.

Polymeric latex dispersed in an aqueous solution was used as an encapsulant because of hydrophobic and latex film forming properties. Since such polymeric latex makes water in the solution evaporate at quite a low energy, it can be used for encapsulation of a biologically active ingredient at a low temperature (Kulvanich, P. and Leesawat, P. 1996, Release characteristics of the matrices prepared from co-spray dried theophylline and ethylcellulose with/without channeling agents. Proceedings of the International Symposium on Controlled Release of Bioactive Materials, 23, 143-144). Latex together with ethylcellulose aqueous polymeric dispersion (Aquacoat™) purchased from FMC Corporation (Philadelphia, USA) was used to prepare an enteric formulation containing a biologically active ingredient by a co-spray drying process (Liao C. W. et al. 2001, Release characteristics of microspheres prepared by co-spray drying Actinobacillus pleuropneumoniae antigens and aqueous ethyl-cellulose dispersion, J. Microencapsulation, Vol. 18, NO. 3, 258-297). In the formulation, detergent is necessary for ethylcellulose to form latex in the aqueous dispersion. However, the detergent causes the biologically active ingredient to be inactive. Taking live microorganisms as an example, when encapsulated in the latex-formed coat, the survival rate of the microorganisms is quite low. Therefore, the polymeric latex dispersed in the aqueous solution is not a suitable encapsulant for the biologically active ingredient, especially for live microorganisms.

Given the above, an economical, effective and orally administrative formulation of a biologically active ingredient is desired.

### SUMMARY OF THE INVENTION

It is surprisingly found that powder ethylcellulose, which is a known coating material, can be used for preparing an “enteric” encapsulant for a biologically active ingredient by a co-spray drying process. The invention provides an effective and economical process for preparing an orally administrative formulation for a biologically active ingredient.

One subject of the invention is to provide a method for preparing an orally administrable formulation comprising a biologically active ingredient for controlled release in a neutral or basic environment, which method comprises the steps of:
(a) dispersing powder ethylcellulose with an average diameter of from about 0.1 μm to about 300 μm in an aqueous solution to provide an aqueous dispersion, wherein the aqueous dispersion is substantially free of detergent;
(b) mixing the biologically active ingredient and the aqueous dispersion obtained in step (a) to provide a mixture; and
(c) spray-drying the mixture obtained in step (b) and retaining the mixture for about 10 seconds to about 2 minutes in a drying chamber at a chamber temperature of about 45° C. to about 100° C. to obtain the orally administrable formulation.

Another subject of the invention is to provide an orally administrable formulation prepared by the method as described above.

### BRIEF DESCRIPTION OF TIHE DRAWINGS

FIG. 1. Effects of the temperature setting in a drying tank on yields of various ethylcellulose formulations in the co-spray drying process. Symbols [symbol] : temperature setting at 62.5, 65, 67.5, and 70° C. in the drying tank during spray-dry running, respectively.

FIG. 2. Morphological observation of the encapsulated PS551 (EC-4) after storage for 2 months. The surface of the sample was sputter-coated with gold/palladium using an ion coater (IB-2, Eiko Engineering®, Kobe, Japan) and visualized with a Stereoscan420 (Leica®, Luton, UK).

FIG. 3. Protein release profile of various encapsulated formulations in the dissolution test (n=3).

FIG. 4. Antacid effect on protein release profiles of the encapsulated formulations (n=3).

FIG. 5. Growth curves of the free and encapsulated PS551 (EC12M1) in intestinal fluid.

### DETAILED DESCRIPTION OF THE INVENTION

The invention provides a method for preparing an orally administrable formulation comprising a biologically active ingredient for controlled release in a neutral or basic environment, which method comprises the steps of:
(a) dispersing powder ethylcellulose with an average diameter of from about 0.1 μm to about 300 μm in an aqueous solution to provide an aqueous dispersion, wherein the aqueous dispersion is substantially free of detergent;
(b) mixing the biologically active ingredient and the aqueous dispersion obtained in step (a) to provide a mixture; and
(c) spray-drying the mixture obtained in step (b) and retaining the mixture for about 10 seconds to about 2 minutes in a drying chamber at a chamber temperature of about 45° C. to about 100° C. to obtain the orally administrable formulation.

As used herein, the term “a biologically active ingredient” refers to a biological material or substance with a biological activity in humans or animals. Preferably, the biologically active ingredient according to the invention is pH sensitive, and is absorbed or acts in the intestine. The biologically active ingredient may be absorbed into the capillary in the intestines. The biologically active ingredient according to the invention includes a microorganism, a protein, an enzyme, serum, and the mixture thereof. In a more preferred embodiment, the biologically active ingredient is a microorganism, most preferably, a live microorganism. The live microorganism is optionally pre-treated to lower the toxicity or increase the compatibility. The microorganism may be any microorganism which provides a biological activity, and may be inactivated by heat or a chemical, such as formaldehyde. In one preferred embodiment of the invention, the microorganism is selected from the group consisting of Enterococcus, Escherichia coli, Lactobacillus acidophilus, Lactobacillus pentose, Bacillus subtilis, and the mixture thereof.

As used herein, the term “an orally administrable formulation” refers to a composition for oral administration. In one embodiment of the invention, the orally administrable formulation is used as a vaccine or a pharmaceutical preparation or an oral probiotic supplementation. The term “a vaccine” as used herein refers to an antigenic substance (such as an antigen) for antibody production, which provides an effect in protecting a subject from infection. Usually, vaccines are used for the prophylaxis of epidemic diseases in humans and animals. To maintain the biological activity of the biologically active ingredient such as an antigen in a vaccine, the biologically active ingredient must be encapsulated with an enteric encapsulant to pass by the stomach but release it into the intestines. For example, the antigenic substrate released in the intestines induces the mucosa (such as Peyer's patch) to produce immunoglobulins to provide a preliminary protection from infections. Recently, many studies show that an oral administration is a safe, convenient and economical way to induce immune response. The orally administrable formulations can be added in feeds or fed to animals. It is the most convenient way to induce immune responses in animals in order to protect them from infections.

Preferably, the orally administrable formulation is a solid or a liquid core embedded in a coat, and is in the form selected from the group consisting of a microcapsule, a microparticle, a microsphere, a micrometric or a microbead, a capsule containing microcapsules, and a tablet containing microcapsules.

According to the invention, the biologically active ingredient is encapsulated to form a microcapsule. In an embodiment of the invention, the microcapsule is further embedded in a coat or a unit that is more easily taken. Usually, the particles of the formulation have a diameter from about 1 to about 2,000 μm.

As used herein, the term “controlled release” refers to a condition that a biologically active ingredient is controlled to release in a particular environment. Preferably, the biologically active ingredient is controlled to release in a neutral or basic environment; more preferably, in an enteric environment. As used herein, the term “an enteric environment” refers to the enteric cavity or a physiologically equivalent environment.

As used herein, the term “an encapsulant” refers to a material for encapsulating or coating the biologically active ingredient. The encapsulant has a property of film forming to encapsulate or coat the biologically active ingredient. The encapsulant according to the invention comprises an aqueous ethylcellulose dispersion. In a preferred embodiment, the encapsulant is prepared by dispersing powder ethylcellulose with an average diameter from about 0.1 μm to about 300 μm in an aqueous solution. More preferably, the average diameter of powder ethylcellulose in the dispersion is from about 0.3 μm to about 3 μm. In an embodiment of the invention, the dispersion of ethylcellulose has a viscosity ranging from about 5 to about 105 cps; more preferably, from about 5 to about 24 cps; and most preferably, from about 18 to about 24 cps. According to the invention, the encapsulant provides an effect in controlling the release of the biologically active ingredient in a neutral or basic environment; more preferably, in an enteric environment. According to the invention, the biologically active ingredient is preferably incorporated into an excipient, carrier, or adjuvant. In a preferred embodiment of the invention, the excipient is selected from the group consisting of starch, milk powder, serum, talc, and the mixture thereof. It is surprised to find that such preferred excipient can provide an enteric effect. Furthermore, the encapsulant can protect the biologically active ingredient from degradation or damage at storage or in the stomach. In an embodiment of the invention, the aqueous dispersion further comprises an enteric encapsulant, which allows the biologically active ingredient to be controlled to release in the intestines. Preferably, the enteric encapsulant is selected from the group consisting of cellulose acetate phthalate (CAP), methyl methacrylate methacrylic acid copolymer, hydroxy propyl methyl cellulosephthalate (HPMCP), polyvinyl acetate phthalate (PVAP), and the mixture thereof. According to the invention, the aqueous dispersion preferably comprises a protectant. In a preferred embodiment of the invention, the protectant is selected from the group consisting of glycerol, polyethylene glycol and the derivatives thereof, and the mixture thereof.

In one preferred embodiment of the invention, the aqueous dispersion further comprises an antacid. The antacid is used for helping the orally administrable formulation to resist the acid environment in the stomach.

According to the invention, the step (a) is to provide an aqueous dispersion, which is prepared by dispersing powder ethylcellulose in an aqueous solution, such as water. According to the invention, the preparation of the dispersion is very easy to perform at a low cost.

In the step (b) of the method, the biologically active ingredient and the aqueous dispersion are mixed to obtain a mixture. The biologically active ingredient can be mixed directly or pretreated in accordance different purposes. For instance, a co-spray drying process is used for mixing the biologically active ingredient and the aqueous dispersion. In an embodiment of the invention, the biologically active ingredient is granulated to form a core before encapsulation to prepare a formulation with a large particle size.

In the step (c) of the method, the mixture obtained in step (b) is spray dried for about 10 seconds to about 2 minutes, preferably for about 10 seconds to about 15 seconds in a drying chamber at a chamber temperature of about 45° C. to about 100° C. to obtain the orally administrable formulation. According to the invention, the temperature is not too high, and therefore, the biological activity of the biologically active ingredient is maintained. According to the invention, the chamber temperature varies with the kinds of the biologically active ingredient used. In a preferred embodiment, the chamber temperature ranges from about 45° C. to about 80° C., more preferably, from about 60° C. to about 65° C. In an embodiment of the invention, the mixture may be spun and spray dried by inletting hot air at a temperature of about 50° C. to about 200° C. for maintaining the chamber temperature. Because the mixture contacts with the hot air in a very short time and the water contained in the formulation absorbs latent heat to gasify, the mixture can be dried at a low temperature. Preferably, the mixture is spun at the speed rate of about 10,000 rpm to about 40,000 rpm.

Optionally, the process according to the invention further comprises a step (d) where the orally administrable formulation in step (c) is collected at a temperature of about 15° C. to about 45° C. in an outlet collecting tank.

In one embodiment of the invention, the method may be carried out in a spray dryer, which comprises (a) a heater for heating air; (b) an atomizer for atomizing the mixture to form micro particles; (c) a drying chamber where the wet micro particles contact the hot air to evaporate water in the micro particles and to form dry powder; (d) a cyclone separator for collecting the powder; and (e) a fan for drafting and exhausting air.

The orally administrable formulation prepared by the method according to the invention have the advantages of: (1) providing a resistance to the gastric juice; (2) having the ability to control to release or slow release in the enteric environment; (3) having a good compatibility with the biologically active ingredient and additive; (4) having a stability; (5) forming a continuous film and a capsule after drying; (6) being non-toxic and safe; (7) being at a low cost; and (8) being suitable for applying in granulating and drying. Furthermore, the biologically active ingredient of the formulation according to the invention has a good biological activity because the aqueous dispersion is substantially free of detergent.

The following Examples are given for the purpose of illustration only and are not intended to limit the scope of the present invention.

### EXAMPLES

Strain: Lactic acid bacteria, Streptococci, were isolated from good-health piglets and a single colony transferred three times in lactobacilli MRS broth (Difico, USA) at 37° C. One isolate, named PS551, was identified as a gram-positive, non-spore forming and facultative anaerobic cocci of the Streptococcaceae family. Using API 20-strep (BioMerieuxe, Inc. Durham, N.C. USA), the identification system for Streptococcaceae and related organisms, PS551 was re-identified as a strain of Enterococcus.

For long term storage of the bacteria isolates, the overnight culture was mixed with an equal volume of 50% sterile glycerol and then stored at −80° C. for the preparation of encapsulated bacteria. 1 ml of glycerol inoculum was incubated in 10 ml MRS broth at 37° C. overnight, and transferred into 200 ml of MRS culture broth in a 500 ml Hinton flask and then cultured at 37° C. for 16 hours. The cultures were collected from MRS broth and stored at 4° C. not more than 3 days before encapsulation.

Preparation of encapsulated bacteria: Encapsulated bacteria were produced by co-spray drying using a Model L-8 (Ohkawara Kakohki Co., Yokohama, Japan) as described by Liao (Liao C. W. et al. 2001). The encapsulated formulations were prepared according to Table 1. The deionized water with various encapsulants or excipients was constantly mixed and stirred. The stirring procedure was maintained for another hour followed by the addition of bacteria broth and then talc. Ordinarily, the process used atomized speed of 26,000 rpm, feeding rate of 1 ml/min, and cyclone pressure of 110 to 120 mm H₂O. The hot-air-inlet or drying tank temperature was varied in different formulations. In the spray-drying process, those encapsulated bacteria were suspended in the drying tank at a temperature (60 to 70° C.) for 20 seconds and then went to the outlet or collection tank.

TABLE 1
Formulation of encapsulated bacteria

| | Deionized | Milk powder | Corn Starch | | | Megadrate | Bacterial broth | |
|---|---|---|---|---|---|---|---|---|
| I.D. | Water (g) | (g) | (g) | MS-1 (g) | ECN14 (g) | (g) | (g) | Talc (g) |
| EC-0 | 100 | 3 | 10 | 20 | 0 | 0 | 400 | 8 |
| EC-0.5 | 100 | 3 | 10 | 20 | 2 | 0 | 400 | 8 |
| EC-1 | 100 | 3 | 10 | 20 | 4 | 0 | 400 | 8 |
| EC-2 | 100 | 3 | 10 | 20 | 8 | 0 | 400 | 8 |
| EC-4 | 100 | 3 | 10 | 20 | 16 | 0 | 400 | 8 |
| EC-8 | 100 | 3 | 10 | 20 | 32 | 0 | 400 | 8 |
| EC-12 | 100 | 3 | 10 | 20 | 48 | 0 | 400 | 8 |
| EC-24 | 100 | 3 | 10 | 20 | 96 | 0 | 400 | 8 |
| EC-4d | 100 | 3 | 10 | 0 | 16 | 0 | 400 | 8 |
| EC-8d | 100 | 3 | 10 | 0 | 32 | 0 | 400 | 8 |
| EC-12d | 100 | 3 | 10 | 0 | 48 | 0 | 400 | 8 |
| EC-12 M0.3 | 100 | 3 | 10 | 20 | 48 | 1.2 | 400 | 8 |
| EC-12 M1 | 100 | 3 | 10 | 20 | 48 | 4 | 400 | 8 |
| EC-12d M1 | 100 | 3 | 10 | 0 | 48 | 4 | 400 | 8 |
| AQ6 | 100 | 3 | 10 | 0 | 60 | 4 | 400 | 8 |

MS-1 is a modified food starch derived from waxy maize especially suited for the encapsulation and purchased from National Starch and Chemical Corporation (Bridgewater, N.J., USA).

Aqualon(& Ethylcellulose type ECN14, another encapsulatant purchased from Herculesg Inc (Wilmington, Del., USA), was added in various amounts to EC-0.5, and EC-1 to EC-16 formulations.

Magaldrate (M) was antacid obtained from Standard Chem. & Pharm. Co., LT. Taiwan).

AQ6 was latex together with ethylcellulose aqueous polymeric dispersion, Aquacoat™.

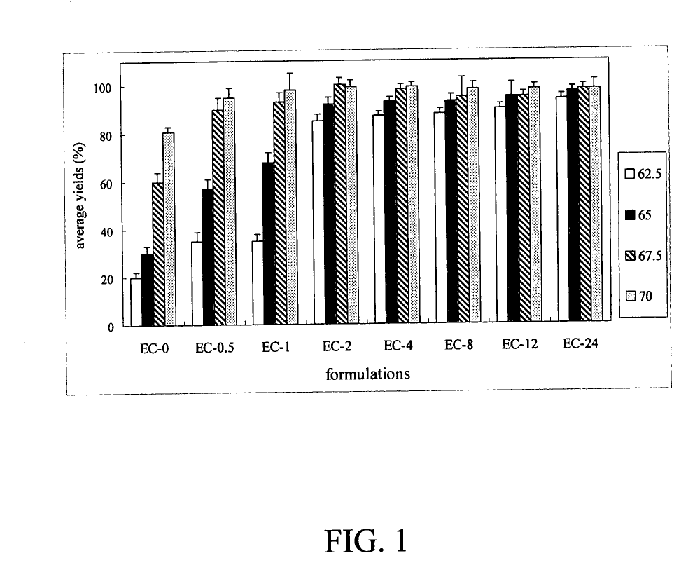

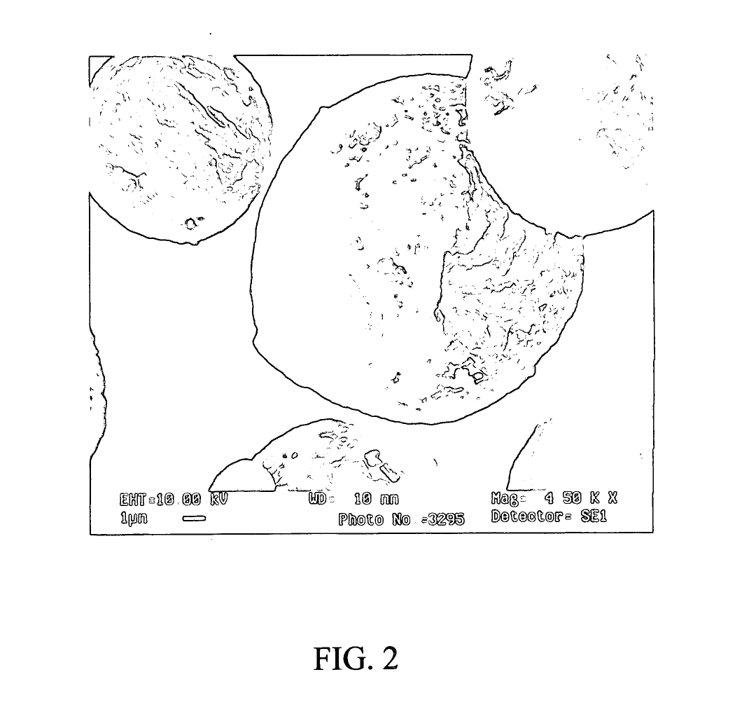

The effects of the temperature setting in the drying tank on yields of various ethylcellulose formulations by the co-spray drying process are shown in FIG. 1. The encapsulated PS551 samples were coated with gold/palladium after storage for 2 months and analyzed by scanning electron microscopy (referring to FIG. 2). It is found that nearly 99% of the PS551 bacterial culture was killed with the formulation of latex form of Aquacoat™ (AQ6) used. On the contrary, all the powder form has a good viability after the spray drying process. For the preparation of the formulation EC-0 without ethylcellulose, the drying tank temperature has to be adjusted to a temperature higher than 70° C. The optimal drying tank temperature for EC-0.5 or EC-1 preparation is at a lower temperature, around 67.5° C. The tank temperature for EC-2, 4, 8, 12, or 24 preparation can be adjusted to 62 to 65° C. In the optimal condition, the microencapsulated samples of approximately 4 to 10 μm in diameter were prepared (referring to FIG. 2).

In vitro protein release study: Proteins such as milk or bacterial cells were the major component in the formulations. The fact that the release characteristics varied with the formulations could be demonstrated by the protein release profile. The dissolution test of encapsulated PS551 was conducted according to the pH changing method of USP-XXI A (United States Pharmacopoeia XXII/NF-XII 1990). In general, 2 g encapsulated PS551 were placed into a jacketed vessel containing 500 ml of 0.03 N HCl solution (pH1.5) at 37° C. and 100 rpm. 20 ml of 0.5 M tri-sodium phosphate buffer was added to adjust the pH to 7 for a further dissolution experiment. During the dissolution test, 1.5 ml of samples were taken out at 30 minutes or 1 hour intervals and 1.5 ml of buffer solution was added to maintain the volume of the dissolution vessel. The samples were centrifuged at 15,000 g for 5 minutes, and the supernatants were aspirated and stored at −20° C. The amount of protein released was determined by a Coomassie protein assay (Liao C. W. et al. 2001).

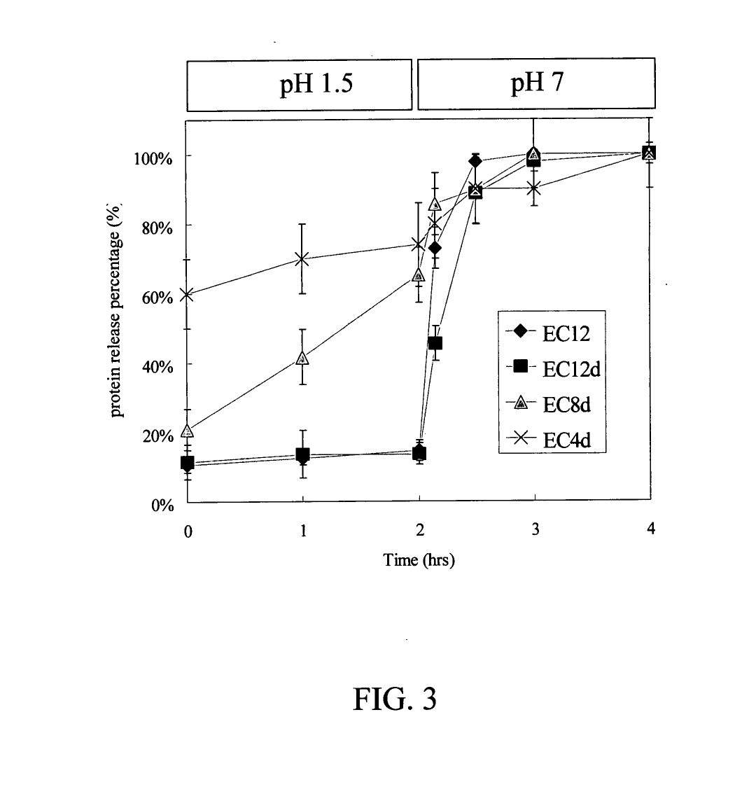

The results are shown in FIG. 3.

In vitro acid resistant and viability study: Acid resistance of the encapsulated formulation was also analyzed according to the pH changing method of dissolution test. 2 g of the encapsulated bacteria were placed in a sterile jacketed beaker containing 500 ml of 0.03 N or 0.01 N HCl solution (pH1.5) at 37° C. and stirred at 100 rpm for 2 hours. 20 or 5 ml of 0.2 M tri-sodium phosphate buffer was added to adjust the pH to 7 for 1 hour. For viable bacterial cfu, 1.5 ml of samples were taken out from vessels after changing to the neutral pH and directly diluted in sterile PBS by tenfold serial dilution and then plated in triplicate onto an MRS-stm plate (MRS medium with 30 ppm streptomycin) and cultured at 37° C. for 2 days. The counts were determined by using the dilution that produced between 30 and 300 cfu per plate.

The viability of bacteria after spray drying preparation or long-period storage was also analyzed using a dissolution test machine. The samples were mixed individually with 500 ml sterile PBS in vessel, and then subjected to the survival bacteria count by the tenfold serial dilution method. In general, 2 g encapsulated bacteria were placed into a sterile jacketed beaker containing 500 ml of PBS solution (pH 7) at 37° C. and stirred for 1 hour at 100 rpm. For viable bacterial cfu, 1.5 ml of samples were taken out and detected by tenfold serial dilution in sterile PBS and then plated in triplicate onto a MRS-stm plate.

The results are shown in Table 2.

TABLE 2
Encapsulation efficacy of encapsulated bacteria
prepared by various formulations (n = 3).

| Formulation differences | | | Encapsulation process | | |
|---|---|---|---|---|---|
| | CAP- | Ethyl- | | | Survival |
| | SULE | cellu- | Average | Viable | rate in |
| | Starch | lose | yields | count | 1 g |
| I.D. | (g) | (g) | (%) | (cfu/g) | powder (%) |
| EC-0 | 20 | 0 | 81 ± 4 | 6.4 ± 0.2 × 10¹⁰ | 45 |
| EC-0.5 | 20 | 2 | 90 ± 3 | 1.3 ± 0.2 × 10¹¹ | 92 |
| EC-1 | 20 | 4 | 93 ± 5 | 1.3 ± 0.2 × 10¹¹ | 96 |
| EC-2 | 20 | 8 | 97 ± 6 | 1.4 ± 0.4 × 10¹¹ | 100 |
| EC-4 | 20 | 16 | 98 ± 2 | 1.3 ± 0.4 × 10¹¹ | 98 |
| EC-8 | 20 | 32 | 99 ± 4 | 1.3 ± 0.4 × 10¹¹ | 98 |
| EC-12 | 20 | 48 | 98 ± 2 | 1.3 ± 0.4 × 10¹¹ | 98 |
| EC-24 | 20 | 96 | 99 ± 3 | 9.1 ± 0.4 × 10¹⁰ | 65 |
| EC-4d | 0 | 16 | 98 ± 5 | 1.2 ± 0.3 × 10¹¹ | 86 |
| EC-8d | 0 | 32 | 93 ± 3 | 1.2 ± 0.6 × 10¹⁰ | 86 |
| EC-12d | 0 | 48 | 91 ± 3 | 9.8 ± 0.5 × 10¹⁰ | 70 |
| AQ6 | 0 | 18 | 95 ± 3 | 1.3 ± 0.4 × 10⁹ | 1 |

CAPSULE: a modified food starch derived from waxy maize.
Average yields: (weight of encapsulated bacteria yield/solid content of total bacterial broth and excipients) × 100%.
Viable count: one gram encapsulated bacteria could be prepared from 6 ml of PS551 bacterial culture broth (1.4 ± 0.3 × 10¹¹ cfu/6 ml). Therefore, the survival rate = (viable count of 1 g sample/1.4 ± 0.3 × 10¹¹ cfu) × 100%.

Viable count: one gram encapsulated bacteria could be prepared from 6 ml of PS551 bacterial culture broth (1.4±0.3×10¹¹ cfu/6 ml). Therefore, the survival rate=(viable count of 1 g sample/1.4+0.3×10¹¹ cfu)×100%.

The survivals of PS551 formulated EC-0 without additional ethylcellulose reduced after being prepared by the spray drying process. In the ethylcellulose formula group (EC-0.5, 1, 2, 4, 8, or 12), the survival rate could be improved by the addition of ethylcellulose ECN14. 4% or 2% of ethylcellulose ECN14 was sufficient to encapsulate PS551. The viable bacterial count of EC-24 with 24% of ECN14 dropped to only 65% survival. Therefore, a content of ECN14 greater than 12% did not improve the survival rate of encapsulated PS551.

Modified starch is also important in the formulation for PS551. Without additional starch (EC-4d, EC-8d and EC-12d), the PS551 survival rate apparently dropped around 14% to 30%.

The survival rate of encapsulated bacteria in storage at 4° C. was stable in the long term. The PS551 free bacteria was not resistant to storage; it dropped from 2.4×10¹⁰ to 7×10⁹ cfu/ml after being stored for 2 months. However, the viable count of encapsulated PS551 was very stable and maintained 1.4±0.3×10¹¹ cfu/g. The microencapsulation process would be beneficial for developing PS551 as a probiotic product.

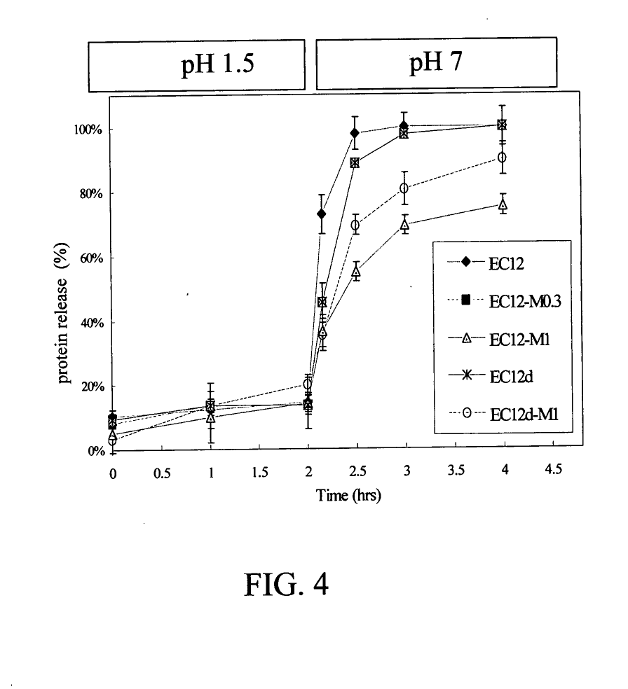

The results of the protein release profile in the acid environment are shown in FIGS. 3 and 4 and Table 3. Unfortunately, the modified starch-coated or ethylcellulose-coated PS551, including EC-0, EC-0.5, EC-2, EC4, EC-8, and EC12, did not display any enhanced viability compared with the free PS551 (shown in Table 3). For further improvement in acid resistance in encapsulated formulations, inorganic antacid: MagaldrateTM (Standard Chem.& Pharm. Co., LT. Taiwan) was added into the formulations of EC-12M1 and EC-12M0.3. The effect of MagaldrateTM concentration on the viability of encapsulated PS551 in a simulated gastric condition (pH1.5 0.01N or 0.03N HCl) for 2 hours at 37° C. was shown in Table 3. This significant reduction of bacterial viability at pH<2 could be reversed when PS551 were encapsulated in the formulation with Magaldrate™ additives. The viability of the encapsulated bacteria in the acid condition increased with increased Magaldrate™ concentration from 0.3% to 1.0% (w/v).

TABLE 3
The antacid additive effect on encapsulated bacteria against gastric acid (HCl)

| The amounts of three excipients in various formulations | | | | Encapsulated bacterial survival viable count (cfu/g) | | | |
|---|---|---|---|---|---|---|---|
| for 400 ml PB551 culture batch | | | | in 0.03 N HCl | | in 0.01 N HCl | |
| | CAPSULE | Ethyl- | Magaldrate | | Survival rate | | Survival rate |
| I.D. | (g) | cellulose (g) | (g) | cfu/g | (%) | cfu/g | (%) |
| EC-0 | 20 | 0 | 0 | 2.4 ± 0.2 × 10⁶ | 0.02 | 3.4 ± 0.2 × 10⁶ | 0.02 |
| EC-1 | 20 | 4 | 0 | 3.6 ± 0.7 × 10⁸ | 0.3 | 9.2 ± 0.3 × 10⁸ | 0.6 |
| EC-2 | 10 | 8 | 0 | 5.4 ± 0.4 × 10⁸ | 0.4 | 7.5 ± 0.6 × 10⁹ | 8 |
| EC-4 | 10 | 16 | 0 | 7.2 ± 0.3 × 10⁸ | 0.7 | 1.4 ± 0.3 × 10¹⁰ | 10 |
| EC-12 | 20 | 48 | 0 | 1.5 ± 0.3 × 10⁹ | 1 | 1.9 ± 0.6 × 10¹⁰ | 11 |
| EC-12d | 0 | 48 | 0 | 9 ± 0.1 × 10⁸ | 0.6 | 3.6 ± 0.1 × 10⁹ | 3 |
| EC-12 M1 | 20 | 48 | 4 | 4.6 ± 0.9 × 10¹⁰ | 33 | 1.4 ± 0.2 × 10¹¹ | 99 |
| EC-12 M0.3 | 20 | 48 | 1.2 | 1.4 ± 0.6 × 10¹⁰ | 10 | 9.8 ± 0.2 × 10¹⁰ | 70 |
| EC-12d M1 | 0 | 48 | 4 | 2.2 ± 0.9 × 10¹⁰ | 16 | 1.3 ± 0.4 × 10¹¹ | 93 |

Encapsulated bacterial survival: After being treated with HCl for 2 hours and then changing the pH to 7 for 1 hour, the sample was taken out for the viable count assay. As shown in Table 2, 1 gram encapsulated bacteria could be prepared from 6 ml of PS551 bacterial culture broth (1.4±0.3×10¹¹ cfu/6 ml). Therefore, the survival rate % was (viable count of 1 g sample/1.4±0.3×10¹¹ cfu)×100%.

Growth of encapsulated PS551 in intestinal fluid: SPF pigs (Landrace) which were at the age 4 weeks after weaning and fed a standard pig diet were chosen randomly from SPF farm. The pigs were killed via a lethal injection of pentobarbital sodium (200 g/liter). Immediately following slaughter, 10 ml to intestinal fluids were drawn from the ileum segment (30 cm from the ileocecal).

The segment was washed again with 10 ml of sterile saline (0.9% NaCl). After mixing these two juices together, the intestinal fluid was homogenized on ice using a homogenizer (polytron pt-300, Kinematica AG) and then filtrated with a 0.2 um MillexGP™ filter unit (Millipore Corningtwohill, Ireland). The sterile fluid was is stored in a −70° C. freezer before performing a bacterial growth experiment. The growth of encapsulated PS551 was compared to the free PS551 or E. coli K88.

Furthermore, the free PS551 or E. coli was respectively plated on a MRS-stm or EMB-stm agar plate and incubated overnight at 37° C. Two colonies were transferred into a 5 ml sterile saline tube and then adjusted to 2×10⁵ cfu/100 μl inoculum through tenfold dilution. PS551 encapsulated with EC-12M1 formulation was aspirated from a dissolution vessel in which encapsulated bacteria with 0.01N HCl had previously been reacted for 2 h followed by the neutral pH (pH 7) for 1 hr at 37° C. during the dissolution test. PS551 from the reacted vessel was diluted to 2×10⁵ cfu/100 μl. One hundred μl dilution above inoculated bacteria were cultured, respectively, in 1 ml intestine fluid and incubated for 24 hours at 37° C. Samples were taken out at 2-hour intervals. The tenfold serial dilution was performed and then the bacteria were counted onto the MRS-stm or EMB-stm plate to investigate bacterial growth.

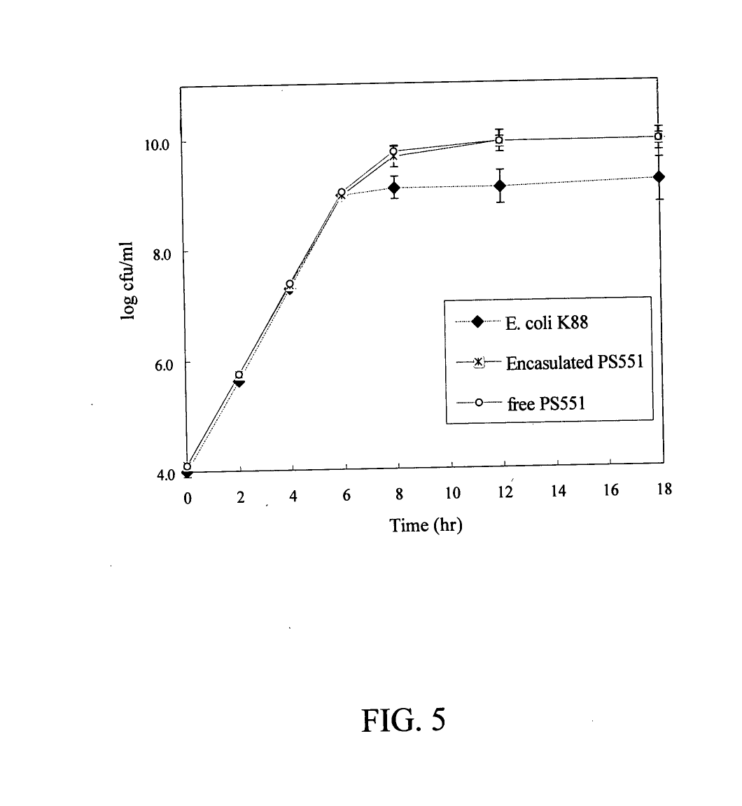

The results are shown in FIG. 5. It demonstrates that the live encapsulated PS551 has the same growth profile of the free PS551. The encapsulated or free PS551 grew well in the early growth phase in intestinal fluid. Its doubling time was 22.4 or 22 min in the log growth phase, respectively. However, the doubling time of E. coli K88 was only 21.7 min.

In vivo feeding trials on pigs challenge model: 2-week-old SEW piglets obtained from SPF farm at Taiwan were distributed among three experiment (2 pigs per group): (i) piglets were orally administered with 10 g of 10⁸ CFU/g encapsulated PS551/dispersed in 60 ml 0.2% acetate water or (ii) 60 ml of free PS551 culture broth or (iii) control group, no probiotics. After being administered once per day for one week, all piglets were challenged intragastrally with 50 ml of 6×10⁸ cfu/ml E. coli K88. Four days after the challenge, the piglets were slaughtered. To monitor the probiotic effect, viable bacterial counts of the feces and intestinal fluid samples were analyzed using the tenfold serial dilutions method, and plated in triplicate onto the appropriate selective media.

Table 4 lists the results of the challenge test in the experimental groups. All piglets in the control group and the free bacteria PS551 group had diarrhea 2 days after the challenge. On the contrary, there were no diarrheal symptoms in the encapsulated PS551 peroral group. It showed that encapsulated PS551 had the ability to protect piglets against E coli K88. The PS551 bacteria seemed to be strong enough to colonize in piglet's intestines. As shown in Table 4, Enterococcus bacteria had 4.3×10⁸ cfu/g in the intestines and 5×10⁷ cfu/g in the feces samples in encapsulated PS551 group after 4 days after the challenge of E. coli K88. Only a small amount of E. coli (400±50 cfu/g) was found in their intestines. In the control group, the amounts of E. coli cfu increased 2.1×10⁶ in the intestines and 4.4×10⁷ in the feces. In the free PS551 orally administrated group, the amount of PS551 cfu only increased a very small amount in the intestines. Without encapsulation, it was difficult for the free PS551 to be delivered into the intestines. From the above experiments, it cab be confirmed that the encapsulated PS551 provided a good protection from diarrhea disease and increased E coli K88 clearance from the intestine.

TABLE 4
Protection of EC-12M PS551 administrated to piglets post-challenge against E. coli K88 isolate (P-203)

| | | | Total | | Ratio of |
|---|---|---|---|---|---|
| | Pathological symptom | | Streptococcus | | Streptococcus/E |
| Groups | Post-challenge | Specimen Sources | (cfu/g) | E coli (cfu/g) | coli cfu |
| Encapsulated PS551 Group | No symptoms | Intestines | 4.3 ± 2 × 10⁸ | 400 ± 59 | 1000000:1 |
| | | Feces | 5 ± 1 × 10⁷ | 6.2 ± 4.2 × 10⁵ | 81:1 |
| Free PS551 Group | Dysentery | Intestines | 8.2 ± 5.6 × 10⁶ | 1.5 ± 1.2 × 10⁵ | 55:1 |
| | | Feces | 5 ± 4 × 10⁶ | 9.5 ± 3.3 × 10⁶ | 0.52:1 |
| Negative control | Dysentery | Intestines | 3.4 ± 1 × 10⁵ | 2.1 ± 0.5 × 10⁶ | 0.16:1 |
| | one dead | Feces | 2.6 ± 1 × 10⁶ | 4.4 ± 2.2 × 10⁷ | 0.06:1 |

While embodiments of the present invention have been illustrated and described, various modifications and improvements can be made by persons skilled in the art. It is intended that the present invention is not limited to the particular forms as illustrated, and that all the modifications not departing from the spirit and scope of the present invention are within the scope as defined in the appended claims.

What is claimed is:

1. A method for preparing an orally administrable formulation comprising a biologically active ingredient for controlled release in a neutral or basic environment, which comprises the steps of:
(a) dispersing powder ethylcellulose with an average diameter of from about 0.1 μm to about 300 μm in an aqueous solution to provide an aqueous dispersion, wherein the aqueous dispersion is substantially free of detergent;
(b) mixing the biologically active ingredient and the aqueous dispersion obtained in step (a) to provide a mixture; and
(c) spray-drying the mixture obtained in step (b) for about 10 seconds to about 2 minutes in a drying chamber at a chamber temperature of about 45° C. to about 100° C. to obtain the orally administrable formulation.

2. The method according to claim 1, wherein the powder ethylcellulose in the aqueous dispersion has a viscosity ranging from about 5 to about 105 cps.

3. The method according to claim 1, wherein the powder ethylcellulose in the aqueous dispersion has a viscosity ranging from about 5 to about 24 cps.

4. The method according to claim 1, wherein the powder ethylcellulose in the aqueous dispersion has a viscosity ranging from about 18 to about 24 cps.

5. The method according to claim 1, wherein the average diameter of the powder ethylcellulose is from about 0.3 μm to about 3 μm.

6. The method according to claim 1, wherein the mixture in step (c) is spray dried for about 10 seconds to about 15 seconds.

7. The method according to claim 6, wherein the chamber temperature is from about 45° C. to about 80° C.

8. The method according to claim 1, wherein the aqueous ethylcellulose dispersion further comprises an enteric encapsulant.

9. The method according to claim 8, wherein the enteric encapsulant is selected from the group consisting of cellulose acetate phthalate (CAP), methyl methacrylate methacrylic acid copolymer, hydroxy propyl methyl cellulosephthalate (HPMCP), polyvinyl acetate phthalate (PVAP), and the mixture thereof.

10. The method according to claim 1 wherein the biologically active ingredient is incorporated into a carrier, adjuvant or excipient.

11. The method according to claim 10, wherein the excipient is selected from the group consisting of starch, milk powder, serum, talc, and the mixture thereof.

12. The method according to claim 1, wherein the aqueous dispersion further comprises a protectant.

13. The method according to claim 12, wherein the protectant is selected from the group consisting of glycerol, polyethylene glycol and the derivatives thereof, and the mixture thereof.

14. The method according to claim 1, wherein the aqueous dispersion further comprises an antacid.

15. The method according to claim 1, wherein the biologically active ingredient is selected from the group consisting of a microorganism, a protein, an enzyme, a serum, and the mixture thereof.

16. The method according to claim 15, wherein the microorganism is selected from the group consisting of Enterococcus, Escherichia coli, Lactobacillus acidophilus, Lactobacillus pentose, Bacillus subtilis, and the mixture thereof.

17. The method according to claim 15, wherein the microorganism is live.

18. The method according to claim 1, wherein the orally administrative formulation is a vaccine.

19. The method according to claim 1 wherein the aqueous solution is water.

20. The method according to claim 1, wherein the biologically active ingredient in step (b) is granulated.

21. The method according to claim 1, wherein the chamber temperature in step (c) is from about 60° C. to about 65° C.

22. The method according to claim 1, wherein the mixture is spray dried by further spinning the mixture at the speed rate of about 10,000 rpm to about 40,000 rpm.

23. The method according to claim 1, wherein the mixture is spray dried by inletting hot air at a temperature from about 50° C. to about 200° C.

24. The method according to claim 1 further comprising a step (d), collecting the orally administrable formulation in step (c) at a temperature of about 15° C. to about 45° C. in an outlet collecting tank.

25. An orally administrative formulation comprising a biologically active ingredient prepared by the method according to claim 1.

26. The formulation according to claim 25, wherein the biologically active ingredient is controlled to release in an enteric environment.

27. The formulation according to claim 25, which is in the form selected from the group consisting of a microcapsule, a microparticle, a microsphere, a micromatrice or a microbead, a capsule containing microcapsules, and a tablet containing microcapsules.
